(19) United States
(12) Patent Application Publication
(10) Pub. No.: US 2026/0209863 A1
(43) Pub. Date: Jul. 23, 2026

(54) DIAGNOSIS OF TUBERCULOSIS IN SALIVA

(71) Applicants: AARHUS UNIVERSITET, Aarhus C (DK); REGION MIDTJYLLAND, Viborg (DK); VPCIR.COM APS, Hørsholm (DK)

(72) Inventors: Kamilla VANDSØ PETERSEN, Aarhus C (DK); Christian Morberg WEJSE, Aarhus C (DK); Birgitta R. KNUDSEN, Aarhus C (DK)

(21) Appl. No.: 19/126,721

(22) PCT Filed: Nov. 3, 2023
(86) PCT No.: PCT/EP2023/080683

§ 371 Date: May 2, 2025

(30) Foreign Application Priority Data
Nov. 4, 2022 (EP) 22205561.8

(51) Int. Cl. C12Q 1/689

(52) U.S. Cl. CPC C12Q 1/689; C12Q 2600/156

(57) ABSTRACT

The present invention relates to diagnosis of tuberculosis in saliva. In particular, the present invention relates to a method for determining whether a subject is likely to be infected with Mycobacterium tuberculosis (mt) by incubating a saliva sample from a subject with a single stranded DNA oligonucleotide comprising an STS binding element for mt DNA Topoisomerase I enzyme (mTOPI) and subsequently determining the level of circularized oligonucleotide. Furthermore, the present invention relates to oligonucleotides comprising an STS binding element and uses thereof.

### TECHNICAL FIELD OF THE INVENTION

The present invention relates to diagnosis of tuberculosis in saliva. In particular the present invention relates to a method for determining whether a subject is likely to be infected with Mycobacterium tuberculosis (mt) by incubating a saliva sample from a subject with a single stranded DNA oligonucleotide comprising an STS binding element for mt DNA Topoisomerase I enzyme (mTOPI) and subsequently determining the level of circularized oligonucleotide. Furthermore, the present invention relates to oligonucleotides comprising an STS binding element and uses thereof.

### BACKGROUND OF THE INVENTION

Tuberculosis (TB) is a serious infectious disease affecting health worldwide. TB is caused by members of the Mycobacterium tuberculosis complex (MTC) and is one of the leading causes of death caused by a single infectious agent. Even though TB is preventable and curable, it remains a threat to global health, as diagnosis and treatment are not always accessible to everyone. Moreover, there is an increase in the development of antimicrobial resistant mycobacteria, which are currently responsible for a quarter of the deaths caused by TB. According to the latest WHO report for 2020, an estimated number of 10 million people were infected with TB and around 1.5 million died. Furthermore, WHO estimates that one quarter of the world are infected with TB, primarily with a latent infection, which in around 10% will progress to active TB. TB is most prevalent in South-East Asia, Africa and the Western Pacific and continues to afflict many countries due to inadequate health care systems and lack of diagnosis caused by e.g. initial mild symptoms of TB, economics and logistics. From the 10 million people estimated to be infected with TB only 5.8 million were diagnosed in 2020. This ultimately results in under-treatment, increased spreading of the disease and more deaths. Hence, improving TB diagnostics is an important tool to decrease the large burden of TB worldwide.

Accurate diagnosis of TB is troublesome as a cause of slow-growing mycobacteria, requirement of difficult obtainable test material from the lungs (called sputum) and the low resources in high TB burden areas. Diagnosis are currently based on primarily sputum smear microscopy and the GeneXpert polymerase chain reaction (PCR) amplification test. The gold standard and most sensitive method is cultivation of the mycobacteria. This can, however, take from 2-8 weeks and is not convenient to use for first line diagnostics. Sputum smear microscopy remains one of the most used methods in low-resource areas as it is rapid, simple and inexpensive, but the method suffers from low sensitivity and specificity and difficulties in obtaining sputum samples from some patients. An alternative is the WHO endorsed GeneXpert MTB/RIF PCR test from Cepheid, which is simple, fast and has a sensitivity and specificity of 85% and 98% in high resource settings. Moreover, the method can detect antimicrobial resistance towards the most used treatments. The method does, however, require a specialized expensive PCR machine and the cost per test is high (>10$). Moreover, the existing techniques require sputum of good quality as test material. Sputum can be troublesome to work with and difficult to obtain from children and patients already diagnosed with HIV. There is a need for fast, simple, and cheap tests, which are point-of-care friendly, and do not require trained staff and stable electricity. More importantly, the tests should be based on non-invasive samples (such as saliva) and not on sputum to improve diagnosis of i.e. children.

### SUMMARY OF THE INVENTION

The invention presented here is a method for diagnosis of TB in saliva samples (not sputum). The method is based on measuring the activity of a pathogen expressed biomarker, namely the Mycobacterial topoisomerase I (mTOPI). mTOPI is an ubiquitously expressed enzyme essential for mycobacterial survival and is thus a good marker for TB infections. mTOPI is a specific enzyme that cleaves within a specific recognition site, the strong topoisomerase site (STS), with the sequence CG/TCT*TC/G (* marks the site of cleavage). mTOPI is able to cleave and ligate single stranded DNA at this site, which is utilized in this method with a specific DNA substrate containing the STS. mTOPI is able to convert the substrate to a circular product that can be amplified with rolling circle amplification (RCA) as in the previous paper by Franch et al. [1]. The final RCA product can be visualized with a fluorescent readout by using fluorescent probes, by incorporating fluorescent nucleotides, or by incorporating biotin-labelled nucleotides that can be coupled to an anti-biotin antibody conjugated to horseradish peroxidase (HRP) for a chemiluminescent or colorimetric readout.

Example 1 illustrates the method of the invention. See also FIG. 1.

Example 2 shows that it is indeed possible to detect TB in saliva samples from subjects infected with TB. The presence of members of the Mycobacteria tuberculosis complex (that is the causative agent of TB in humans) in saliva of infected individuals in sufficient concentration has not previously been demonstrated and diagnosis using this test material is not obvious. Indeed, the standard for TB diagnosis is testing in sputum, which is known to contain the highest concentration of the Mycobacteria tuberculosis complex.

Example 3 shows that the previous used oligonucleotide substrate can be optimized to improve detection of TB. This is achieved by the use of oligonucleotides with all the possible combinations of the STS sequence. Moreover, a pseudo-STS sequence in the region complementary to the primer has been changed and shown to improve the detection efficiency.

Example 4 shows that the one-step protocol, wherein Mg²⁺ ions is also present during the cleavage of the oligonucleotides, was simpler, faster and increased the sensitivity of the method without compromising the specificity as compared to the two-step protocol, wherein Mg²⁺ ions is only added after cleavage prior to the ligation step. Thus, to improve the detection limit TB-EAD and allow detection of mycobacteria in saliva samples, Mg²⁺ is added to both the mTOP1 cleavage and ligation step of the procedure (one combined step). This improved detection limit, which is not obvious since the addition of Mg²⁺ enables the activity of unspecific nucleases that compete with mTOP1 for reaction with the substrate and, hence, may hamper detection of mTOP1 activity.

Example 5 shows that freeze/thawing combined with vortexing appears to be the most effective protocol for lysis of M. tuberculosis samples whereas bead-beating was rather effective for lysis of M. smegmatis.

Thus, an object of the present invention relates to the provision of an improved method for detection TB in saliva samples. Another object of the present invention relates to the provision of improved oligonucleotides substrates, which can improve detection in methods according to the invention.

In particular, it is an object of the present invention to provide a method that solves the above mentioned problems of the prior art with efficient detection of TB in saliva samples.

Thus, one aspect of the invention relates to a method for determining whether a subject is likely to be infected with Mycobacterium tuberculosis, the method comprising
a) having a (previously obtained) saliva sample from a subject provided;
b) providing a single stranded DNA oligonucleotide (1) comprising:
   a binding element (Strong Topoisomerase Site; STS) for Mycobacterium tuberculosis DNA Topoisomerase I enzyme (mTOPI) (2);
c) incubating the saliva sample of step a) with the oligonucleotide (1) from step b), wherein if mTOPI (2) is present in the sample of step a), the single stranded DNA oligonucleotide of step b) is circularized (3);
d) determining the level of circularized oligonucleotide in the sample from step c);
e) comparing said determined level to a reference level; and
f) determining that
   if said level is above said reference level it is indicative of said subject being infected with Mycobacterium tuberculosis; or
   if said reference level is equal to or below said reference, it is indicative of said subject not being infected with Mycobacterium tuberculosis.

An aspect also relates to a method for determining whether a subject is likely to be infected with Mycobacterium tuberculosis, the method comprising
a) having a saliva sample from a subject provided;
b) providing a combination of types of single stranded DNA oligonucleotides (1) comprising:
   a binding element (Strong Topoisomerase Site; STS) for Mycobacterium tuberculosis DNA Topoisomerase I enzyme (mTOPI) (2);
   a primer-annealing element, complementary to a DNA oligonucleotide (4);
   wherein the single stranded DNA oligonucleotides (1) are attached to a solid support (5) via the DNA oligonucleotide (4);
c) incubating the saliva sample of step a) with the oligonucleotides (1) from step b), wherein if mTOPI (2) is present in the sample of step a), the single stranded DNA oligonucleotides of step b) are circularized (3);
d) determining the level of circularized oligonucleotides in the sample from step c);
e) comparing said determined level to a reference level; and
f) determining that
   if said level is above said reference level it is indicative of said subject being infected with Mycobacterium tuberculosis; or
   if said reference level is equal to or below said reference, it is indicative of said subject not being infected with Mycobacterium tuberculosis;
      wherein in step b) the types of single stranded oligonucleotides (1) comprise different binding elements selected from 5′-CGCTTG-3′, 5′-CGCTTC-3′, 5′-CTCTTG-3′, and 5′-CTCTTC-3′, such as wherein two different types of single stranded oligonucleotides (1) are used, preferably three different types of single stranded oligonucleotides (1) and more preferably four different types of single stranded oligonucleotides (1);
      wherein in step c) Mg²⁺ ions are present;
      wherein in step d) the level of circularized DNA (3) of step d) is determined using a method selected from the group consisting of Rolling Circle Amplification (RCA), PCR, real-time-PCR, Southern blotting, quantitative PCR (qPCR), restriction fragment length dimorphism-PCR (RFLD-PCR), primer extension, DNA array technology, LAMP and isothermal amplification, preferably using RCA (6).

Another aspect of the present invention relates to an isolated single stranded DNA oligonucleotide (1) comprising:
a binding element (Strong Topoisomerase Site; STS) for Mycobacterium tuberculosis DNA Topoisomerase I enzyme (mTOPI) (2) selected from the group consisting of 5′-CGCTTG-3′, 5′-CGCTTC-3′, 5′-CTCTTG-3′, and 5′-CTCTTC-3′, said binding element being located within the first 30 nucleotides, preferably within the first 20 nucleotides of the 5′-end;
optionally a primer-annealing element located after the binding element, when counted from the 5′-end, said primer-annealing element being complementary to a DNA oligonucleotide (4) attached to a solid support (5), such as a glass slide;
optionally, an identification element identical to the sequence of a fluorescently labelled probe (7), said probe preferably being SEQ ID NO: 12;
   wherein the single stranded oligonucleotide (1) is free of a 5′-CGCTT-3′ sequence element after position 25, when counted from the 5′-end, such as after position 30 or after position 35.

Still an aspect relates to a composition comprising two or more types of single stranded oligonucleotides (1) according to the invention; wherein the two or more types have different binding elements (Strong Topoisomerase Site; STS) for Mycobacterium tuberculosis DNA Topoisomerase I enzyme (mTOPI) (2) selected from the group consisting of 5′-CGCTTG-3′, 5′-CGCTTC-3′, 5′-CTCTTG-3′, and 5′-CTCTTC-3′.

Yet another aspect of the present invention relates to the use of an oligonucleotide according to the invention or a composition according to the invention for determining, in a saliva sample, whether a subject is likely to be infected with Mycobacterium tuberculosis (mt).

A related aspect relates to the use of a single stranded DNA oligonucleotide (1) comprising a binding element (Strong Topoisomerase Site; STS) for Mycobacterium tuberculosis DNA Topoisomerase I enzyme (mTOPI) (2) for determining, in a saliva sample, whether a subject is likely to be infected with Mycobacterium tuberculosis (mt).

Still another aspect of the present invention is to provide a kit of parts comprising:
a first container comprising a DNA oligonucleotide (1) according to the invention or a composition according to the invention; and
optionally, instructions for performing a method according to the invention.

### BRIEF DESCRIPTION OF THE FIGURES

FIG. 1 is a depiction of the TB-EAD method. The amine functionalized RCA primer (4) is initially coupled to NHS-ester modified glass slides (5) (step I) and the mTOPI DNA substrates (1) are hybridized to these primers (step II). Mycobacteria present in samples are lysed by freezing and thawing combined with vortexing with glass beads (step III) or infection with lytic mycobacteriophages. The extract is added to the slide and if mTOPI (2) is present in the sample it will cleave and ligate the DNA substrate and generate a closed DNA circle (3) (step IV and V). RCA (6) will generate a long tandem repeat product complimentary to the circle (step VI) which can be visualized with fluorescent probes (7) or by incorporating fluorescent nucleotides in the RCA product (step VII). The fluorescent product can be detected with a fluorescent microscope or a fluorescent scanner (step VIII). Alternatively, biotin-labelled nucleotides (8) can be incorporated in the RCA product (step IX) and bound to an anti-biotin HRP conjugated antibody (9) (step X). By adding TMB or ECL this will result in a colorimetric readout visual to the eye or a chemiluminescent readout that can be detected with a CCD camera or developer.

FIG. 2 shows the results from a Guinea Bissau field study. A) TB-EAD values in patient saliva samples tested either positive or negative for tuberculosis using the GeneXpert MTB/RIF PCR test. The negative samples were collected in Denmark. A line at TB-EAD value 0.22 marks the threshold between positive and negative TB-EAD result and was used for statistical analysis. B) Statistical analyses. Data was analyzed with a Fischer's exact test and the sensitivity and specificity is stated with the 95% confidence interval (CI).

FIG. 3 shows results from comparison of substrates. The number of signals/image frame obtained when comparing different substrate designs. The original mTOPI TP ID33 STS1 (SEQ ID NO: 1) was compared to the Long mTOPI TP ID33 (SEQ ID NO: 2), a mix of substrates with different STS: mTOPI TP ID33 STS2 (SEQ ID NO: 3), mTOPI TP ID33 STS3 (SEQ ID NO: 4), and mTOPI TP ID33 STS4 (SEQ ID NO: 5), and finally to the mTOPI Mus ID33 STS1 substrate (SEQ ID NO: 6) using the Mus RCA primer (SEQ ID NO: 11) instead of the TP RCA primer (SEQ ID NO: 12).

FIG. 4 shows the results from the optimization of the two-step TB-EAD to a one-step protocol with Magnesium. A) Original two-step protocol, B) One-step protocol. Both protocols are done with buffer alone (negative), purified M. smegmatis Topoisomerase I (mTOPI), M. smegmatis, E. coli, E. aerogenes, P. fluorescens, B. megaterium and L. seeligeri. Data is plotted as individual values with mean and standard deviation (SD) of signals/image frame, n=4.

FIG. 5 shows the results from applying different lysis protocols to lyse M. smegmatis and M. tuberculosis. A) Lysis of 200,000 Mycobacterium smegmatis with 3×45 seconds of bead-beating (BB), 3×2 minutes vortexing, or 2 hours with D29 mycobacteriophages (phages). B) Lysis of 800,000 M. tuberculosis with 3×2 or 8×2 minutes (′) vortexing alone, 2× freeze/thawing (F/T)+vortexing, phages+vortexing, and phages alone. Data is plotted as individual values with mean and SD for n=3. For lysis of M. tuberculosis with phages n=2.

FIG. 6 shows the results from lysis of mycobacteria in patient saliva samples. Relative mTOPI activity with mean as measured with TB-EAD normalized to the mTOPI control in patient saliva collected in Guinea Bissau (GB) samples (n=75) lysed by A) freeze/thawing and 3×2 minutes vortexing, B) 3×2 minutes vortexing or C) not lysed, all compared to samples from Denmark (DK) (n=83).

The present invention will now be described in more detail in the following.

### DETAILED DESCRIPTION OF THE INVENTION

### Definitions

Prior to discussing the present invention in further details, the following terms and conventions will first be defined:

### Oligonucleotide

In the present context, an oligonucleotide is a sequence of DNA (or RNA) nucleotide residues that form a molecule. Oligonucleotides can bind their complementary sequences to form duplexes (double-stranded fragments) or even fragments of a higher order. Oligonucleotides can be on a linear form, but also exist as circular oligonucleotide molecules, such as single stranded circular DNAs. When referring the length of a sequence, reference may be made to the number of nucleotide units or to the number of bases. Furthermore, the typical DNA or RNA nucleotides may be replaced by nucleotides analogues such as 2′-O-Me-RNA monomers, 2′-O-alkyl-RNA monomers, 2′-amino-DNA monomers, 2′-fluoro-DNA monomers, locked nucleic acid (LNA) monomers, arabino nucleic acid (ANA) monomers, 2′-fluoro-ANA monomers, 1,5-anhydrohexitol nucleic acid (HNA) monomers, peptide nucleic acid (PNA), and morpholinoes. In the present context, the oligonucleotide should be an enzymatic target for Mycobacterium tuberculosis DNA Topoisomerase I enzyme and enable subsequent amplification by e.g. RCA or PCR. Thus, preferably the oligonucleotide is DNA, but may comprise one or more modified nucleotides. Also preferred is the one or more single stranded oligonucleotides (1) free of modified nucleotides. In the example section, non-modified single stranded DNA oligonucleotides (1) have been used.

### Topoisomerase IA:

Type IA topoisomerases create a single break in DNA and pass a second strand or duplex through the break. This strand passage mechanism shares several features with type IIA topoisomerases. They both form a 5′ phosphotyrosine intermediate, and require a divalent metal ion to perform its work. Unlike type II topoisomerases, type IA topoisomerases do not use energy to do its work (with the exception of reverse gyrase). Like all known type IA topoisomerases, mycobacteria TOPIA regulates DNA topology in a Mg²⁺ dependent reaction that involves temporary nicking of the DNA and the formation of a transient covalent 5′-phosphotyrosyl cleavage complex. Restoration of intact DNA and release of the enzyme is ensured by subsequent ligation of the 3′-OH DNA end formed during cleavage.

### Diagnosis of Tuberculosis in Saliva

As outlined above, the inventing team has conducted clinical trials (see example 2) documenting that Mycobacterium tuberculosis infections can indeed be identified in saliva samples (different from sputum) from infected subjects (and not in uninfected subjects), using the method of the invention. Thus, an aspect of the invention relates to a method for determining whether a subject is likely to be infected with Mycobacterium tuberculosis, the method comprising
a) having a (previously obtained) saliva sample from a subject provided;
b) providing one or more single stranded DNA oligonucleotides (1) comprising:
   a binding element (Strong Topoisomerase Site; STS) for Mycobacterium tuberculosis DNA Topoisomerase I enzyme (mTOPI) (2);
c) incubating the saliva sample of step a) with the one or more oligonucleotides (1) from step b), wherein if mTOPI (2) is present in the sample of step a), the one or more single stranded DNA oligonucleotides of step b) is circularized (3);
d) determining the level of circularized oligonucleotide in the sample from step c);
e) comparing said determined level to a reference level; and
f) determining that
   if said level is above said reference level it is indicative of said subject being infected with Mycobacterium tuberculosis; or
   if said reference level is equal to or below said reference, it is indicative of said subject not being infected with Mycobacterium tuberculosis.

An aspect also relates to a method for determining whether a subject is likely to be infected with Mycobacterium tuberculosis, the method comprising
a) having a saliva sample from a subject provided;
b) providing a combination of types of single stranded DNA oligonucleotides (1) comprising:
   a binding element (Strong Topoisomerase Site; STS) for Mycobacterium tuberculosis DNA Topoisomerase I enzyme (mTOPI) (2);
   a primer-annealing element, complementary to a DNA oligonucleotide (4);
   wherein the single stranded DNA oligonucleotides (1) are attached to a solid support (5) via the DNA oligonucleotide (4);
c) incubating the saliva sample of step a) with the oligonucleotides (1) from step b), wherein if mTOPI (2) is present in the sample of step a), the single stranded DNA oligonucleotides of step b) are circularized (3);
d) determining the level of circularized oligonucleotides in the sample from step c);
e) comparing said determined level to a reference level; and
f) determining that
   if said level is above said reference level it is indicative of said subject being infected with Mycobacterium tuberculosis; or
   if said reference level is equal to or below said reference, it is indicative of said subject not being infected with Mycobacterium tuberculosis;
      wherein in step b) the types of single stranded oligonucleotides (1) comprise different binding elements selected from 5′-CGCTTG-3′, 5′-CGCTTC-3′, 5′-CTCTTG-3′, and 5′-CTCTTC-3′, such as wherein two different types of single stranded oligonucleotides (1) are used, preferably three different types of single stranded oligonucleotides (1) and more preferably four different types of single stranded oligonucleotides (1);
      wherein in step c) Mg²⁺ ions are present;
      wherein in step d) the level of circularized DNA (3) of step d) is determined using a method selected from the group consisting of Rolling Circle Amplification (RCA), PCR, real-time-PCR, Southern blotting, quantitative PCR (qPCR), restriction fragment length dimorphism-PCR (RFLD-PCR), primer extension, DNA array technology, LAMP, and isothermal amplification, preferably using RCA (6).

As shown in example 2 of the present invention, clinical trials have been conducted, documenting that the method of the invention indeed works as claimed.

### Step a)

In a preferred embodiment, the subject is a mammal preferably a human.

In another embodiment, the saliva sample of step a) is a lysed saliva sample.

In a related embodiment, lysis is performed by i) freeze and thawing (e.g. two times of freezing and thawing) the saliva sample, preferably followed by vortexing with a solid material, such as glass beads; ii) bead beating; and/or iii) using lytic mycobacteriophages. In the example section, method i) is used, however, the skilled person may be aware of other methods to lyse the sample. Freezing and thawing may be performed by freezing to −20° C., preferably −80° C., followed by thawing to above 0° C. The cycle can the be repeated two or more times.

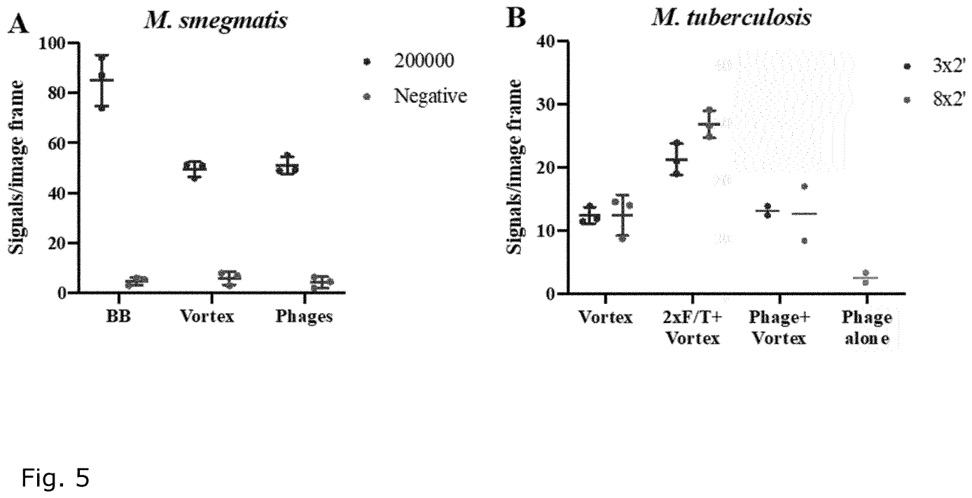

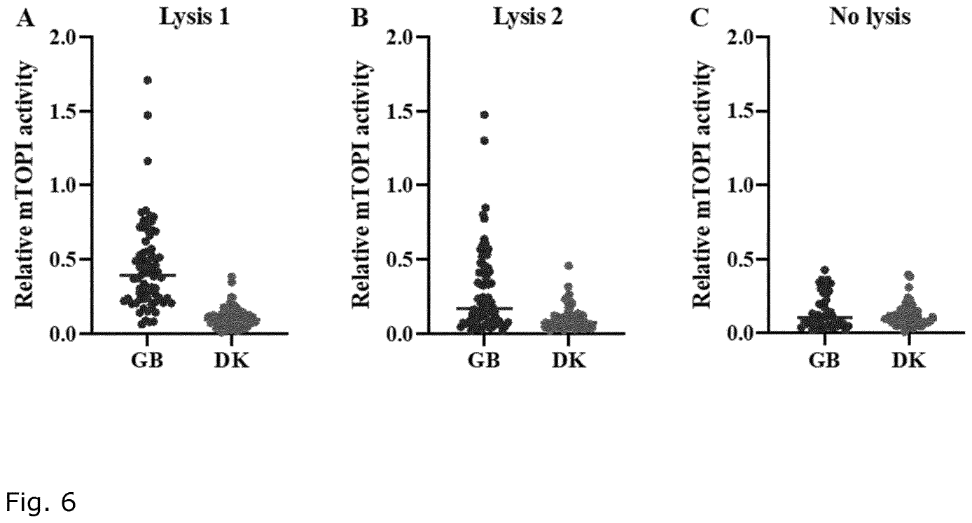

In a preferred embodiment, the lysis is performed by freeze and thawing (e.g. two times of freezing and thawing) the saliva sample before vortexing the sample with a solid material, such as glass beads. In example 5 (FIGS. 5 and 6), it is clear that this protocol is more effective in lysing M. tuberculosis than the other protocols tested.

In an embodiment, the saliva sample is free from sputum or substantially free from sputum.

### Step b)

The mycobacterial TOP1A enzyme has increased binding efficiency to different sequence elements. Thus, in an embodiment, the binding element of the one or more single stranded oligonucleotides (1) of step b) is selected from the group consisting of 5′-CGCTTG-3′, 5′-CGCTTC-3′, 5′-CTCTTG-3′, 5′-CTCTTC-3′ and combinations thereof, preferably comprising combinations of oligonucleotides comprising 5′-CGCTTG-3′, 5′-CGCTTC-3′, 5′-CTCTTG-3′, and 5′-CTCTTC-3′. It is to be understood that each single oligonucleotide (1) comprises one of the four listed sequences. As shown in example 3, it has been found that a combination of oligonucleotides gives the best result. In an embodiment the combination comprises two of 5′-CGCTTG-3′, 5′-CGCTTC-3′, 5′-CTCTTG-3′, and 5′-CTCTTC-3′, such as three of 5′-CGCTTG-3′, 5′-CGCTTC-3′, 5′-CTCTTG-3′, and 5′-CTCTTC-3′ such as all of 5′-CGCTTG-3′, 5′-CGCTTC-3′, 5′-CTCTTG-3′, and 5′-CTCTTC-3′.

Thus, in an embodiment, in step b), a combinations of types of single stranded oligonucleotides (1) are used, comprising different binding elements selected from 5′-CGCTTG-3′, 5′-CGCTTC-3′, 5′-CTCTTG-3′, and 5′-CTCTTC-3′.

In yet an embodiment, two different types of single stranded oligonucleotides (1) are used, preferably three different types single stranded oligonucleotides (1) and more preferably four different types single stranded oligonucleotides (1).

In an embodiment, the binding element of the one or more single stranded oligonucleotides (1) of step b) is located within the first 50 nucleotides, preferably within the first 40 nucleotides, more preferably within the first 30 nucleotides, most preferably within the first 20 nucleotides of the 5′-end.

In another embodiment, the one or more single stranded oligonucleotides (1) of step b) further comprises a primer-annealing element.

In yet another embodiment, the primer-annealing element of the one or more single stranded oligonucleotides (1) of step b) is complementary to a DNA oligonucleotide (4) attached to a solid support (5), such as a glass slide.

In a further embodiment, the oligonucleotide (4) attached to a solid support (5) is modified with an amine, preferably at the 5′-end.

In an embodiment, the primer annealing element of the one or more single stranded oligonucleotides (1) of step b) is at least 25 nucleotides long, preferable at least 20 nucleotides long, more preferably at least 18 nucleotides long.

In an embodiment, the one or more single stranded DNA oligonucleotide (1) of step b) has a length in the range 80-120 nucleotides, such as 90-110, preferably in the range 95-105 nucleotides, such as 98-102.

In an embodiment, the one or more single stranded oligonucleotide (1) of step b) further comprises an identification element.

In yet an embodiment, the identification element of the one or more single stranded oligonucleotides (1) of step b) is identical to the sequence of a fluorescently labelled probe (7), said probe preferably being SEQ ID NO: 12.

In an embodiment, the one or more single stranded oligonucleotide (1) of step b) is selected from the group consisting of SEQ ID NO: 1-9, such as selected from SEQ ID NO: 2-6 or selected from SEQ ID NO: 3-9, preferably selected from SEQ ID NO: 3-6.

In yet an embodiment, the one or more types of single stranded oligonucleotides (1) of step b) is selected from the group consisting of SEQ ID NO: 1-9 or combinations thereof, such as selected from SEQ ID NO: 2-6 or combinations thereof, or selected from SEQ ID NO: 3-9 or combinations thereof, preferably selected from SEQ ID NO: 3-6 or combinations thereof. In an embodiment, a combination of SEQ ID NO: 1+SEQ ID NO: 3-5 is used. In another embodiment, a combination of SEQ ID NO: 6-9 is used.

In an embodiment, at least SEQ ID NO: 4 is used. Preliminary data indicates that this oligonucleotides is better than the others, when used alone (data not shown).

As outlined in example 3, the method can be improved by optimizing the used sequences. Thus, in a preferred embodiment, the one or more single stranded oligonucleotides (1) of step b) is selected from the group consisting of SEQ ID NO: 3-6.

In another preferred embodiment, the one or more single stranded oligonucleotides (1) of step b) is selected from the group consisting of SEQ ID NO: 6-9, more preferably SEQ ID NO: 6.

As also outlined in example 3 a pseudo-binding site has been identified in SEQ ID Nos: 1-5. Removal of such a pseudo-binding site may improve the method. Thus, in an embodiment, the one or more single stranded oligonucleotide (1) is free of a 5′-CGCTT-3′ sequence element after position 25, when counted from the 5′-end, such as after position 30 or after position 35. As shown in example 3, SEQ ID NO: 6 outperforms SEQ ID NO: 1.

It could be foreseen that it would be advantageous to avoid other pseudo-binding sites. Thus, in an embodiment, the one or more single stranded oligonucleotides (1) is free from one or more sequence elements, after position 25 when counted from the 5′-end, selected from the group consisting of:

CGCTT;
CTCTT;
GCTTG;
GCTTC;
TCTTG; and
TCTTC.

### Step c)

In an embodiment, Mg²⁺ ions are added to the mixture of step c) in a reaction environment allowing circularization of the one or more oligonucleotides (1).

In an embodiment, in step c) Mg²⁺ ions are present, such as at a concentration in the range 2-20 mM, preferably 5-15 mM, more preferably 8-12 mM.

In another embodiment, in step c) Mn²⁺ ions are present, such as at a concentration in the range 2-20 mM, preferably 5-15 mM, more preferably 8-12 mM.

Example 4 shows that the one-step protocol, wherein Mg²⁺ ions is also present during the cleavage of the oligonucleotides, was simpler, faster and increased the sensitivity of the method without compromising the specificity as compared to the two-step protocol, wherein Mg²⁺ ions is only added after cleavage prior to the ligation step. Thus, in an embodiment, Mg²⁺ ions are added prior to the cleavage of the oligonucleotide (1) of step b).

Thus, both the cleavage and ligation step take place during step c).

### Step d)

In an embodiment, the level of circularized DNA (3) of step d) is determined using a method selected from the group consisting of Rolling Circle Amplification (RCA), PCR, real-time-PCR, Southern blotting, quantitative PCR (qPCR), restriction fragment length dimorphism-PCR (RFLD-PCR), primer extension, DNA array technology, LAMP, and isothermal amplification, preferably using RCA (6).

In an embodiment, DNA polymerase, and deoxyribonucleoside triphosphates (dNTPs) are added to the incubated mixture of step c) in a reaction environment allowing amplification.

In another embodiment, a portion of the dNTPs, such as all dUTPs, are modified.

In yet another embodiment, the portion of dNTPs being modified, are modified by the addition of a fluorescent label or a biotin group (8).

In an embodiment, amplification of a RCA product is determined by hybridizing a labelled detection oligonucleotide to the RCA product, such as a fluorescently labelled oligonucleotide.

### Step e)

In an embodiment, the reference level of step e) is determined in a subject or a group of subjects without a Mycobacterium tuberculosis infection.

In another embodiment, the reference level of step e) is determined using the same method as used to determine the level of circularized DNA in step d).

In an embodiment, the threshold level is 0.2 TB-EAD value or above, such as above 0.3 or above 0.4. In another embodiment, the threshold level is in the range 0.2-0.6 TB-EAD value, such as in the range 0.2-0 or such as 0.3-0.4. As shown in Example 2, TB-EAD resulted in a sensitivity and specificity of 92.31% and 95.79%, respectively, when setting threshold for a positive TB infection to 0.22 for relative mTOPI activity.

### DNA Oligonucleotides

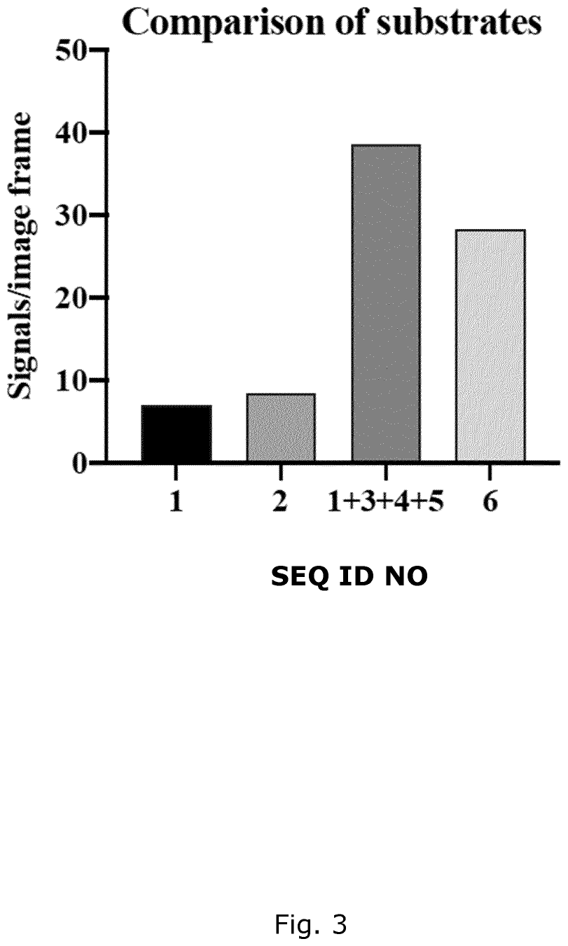

As outlined above, the inventing team has identified optimized oligonucleotides which can find use in a method according to the invention. This is further elaborated in e.g. example 3. Thus, an aspect of the invention relates to an isolated single stranded DNA oligonucleotide (1) comprising:
a binding element (Strong Topoisomerase Site; STS) for Mycobacterium tuberculosis DNA Topoisomerase I enzyme (mTOPI) (2) selected from the group consisting of 5′-CGCTTG-3′, 5′-CGCTTC-3′, 5′-CTCTTG-3′, and 5′-CTCTTC-3′, said binding element being located within the first 30 nucleotides, preferably within the first 20 nucleotides of the 5′-end;
optionally a primer-annealing element located after the binding element, when counted from the 5′-end, said primer-annealing element being complementary to a DNA oligonucleotide (4) attached to a solid support (5), such as a glass slide;
optionally, an identification element identical to the sequence of a fluorescently labelled probe (7), said probe preferably being SEQ ID NO: 12;
   wherein the single stranded oligonucleotide (1) is free of a 5′-CGCTT-3′ sequence element after position 25, when counted from the 5′-end, such as after position 30 or after position 35. Again, as shown in example 3, the presence of the pseudo-binding site appears to limit the efficiency of the assay. Thus, removal of this site improves the assay (See FIG. 3, in particular, compare effect of SEQ ID NO: 1 (comprising the pseudo binding site) and SEQ ID NO: 6 (no pseudo binding site).

In an embodiment, said single stranded DNA oligonucleotide (1) has a length in the range of 80-120 nucleotides, such as 90-110, preferably in the range of 95-105 nucleotides, such as 98-102.

It could be foreseen that it would be advantageous to avoid other pseudo-binding sites. Thus, in an embodiment, the single stranded oligonucleotide (1) is free from one or more sequence elements, after position 25 when counted from the 5′-end, selected from the group consisting of:

CGCTT;
CTCTT;
GCTTG;
GCTTC;
TCTTG; and
TCTTC.

In a preferred embodiment, said oligonucleotide is selected from the group consisting of SEQ ID NO: 6-9, such as being SEQ ID NO: 6.

In a preferred embodiment, the single stranded oligonucleotide is a DNA oligonucleotide. In another preferred embodiment, the single stranded oligonucleotide is free of modified nucleotides, such as 3′ end blocking or 5′ end blockings. Such blockings would normally be introduced to prevent degradation by endogenous nucleases, in particular when Mg is present in the reaction mixture.

Surprisingly the assay works efficiently in the presence of Mg ions without using modified nucleotides (see e.g. example 3 and 4).

### Composition

As outlined in example 3, using a combination of different single stranded oligonucleotides (1) in the method of the invention, stronger signalling are achieved and thus also a more robust and/or sensitive assay. Thus, an aspect of the invention relates to a composition comprising two or more types of single stranded oligonucleotides (1) according to the invention; wherein the two or more types have different binding elements (Strong Topoisomerase Site; STS) for Mycobacterium tuberculosis DNA Topoisomerase I enzyme (mTOPI) (2) selected from the group consisting of 5′-CGCTTG-3′, 5′-CGCTTC-3′, 5′-CTCTTG-3′, and 5′-CTCTTC-3′.

In an embodiment, the composition comprises three or more types of single stranded oligonucleotides (1) according to the invention; wherein the three or more types have different binding elements (Strong Topoisomerase Site; STS) for Mycobacterium tuberculosis DNA Topoisomerase I enzyme (mTOPI) (2) selected from the group consisting of 5′-CGCTTG-3′, 5′-CGCTTC-3′, 5′-CTCTTG-3′, and 5′-CTCTTC-3′.

In a preferred embodiment, the composition comprises four types of single stranded oligonucleotides (1) according to the invention; wherein the four types have different binding elements (Strong Topoisomerase Site; STS) for Mycobacterium tuberculosis DNA Topoisomerase I enzyme (mTOPI) (2) selected from the group consisting of 5′-CGCTTG-3′, 5′-CGCTTC-3′, 5′-CTCTTG-3′, and 5′-CTCTTC-3′.

In an embodiment, the ratio (by concentration) between the two or more single stranded oligonucleotides (1) deviates at the most by 20% from the one with the highest concentration to the one with the lowest concentration, such as deviating at the most by 10%, such as by the most 5%, preferably the concentration for each oligonucleotide is the same. This also applies to the case with three or four single stranded oligonucleotides (1) according to the invention in the composition.

### Uses of Oligonucleotides

An aspect of the invention relates to the use of a single stranded oligonucleotide according to the invention or a composition according to the invention for determining, in a saliva sample, whether a subject is likely to be infected with Mycobacterium tuberculosis (mt).

A related aspect relates to the use of a single stranded oligonucleotide (1) comprising a binding element (Strong Topoisomerase Site; STS) for Mycobacterium tuberculosis DNA Topoisomerase I enzyme (mTOPI) (2) for determining, in a saliva sample, whether a subject is likely to be infected with Mycobacterium tuberculosis (mt).

In an embodiment, the STS is selected from the group consisting of 5′-CGCTTG-3′, 5′-CGCTTC-3′, 5′-CTCTTG-3′, and 5′-CTCTTC-3′.

In another embodiment, the STS of the single stranded oligonucleotide (1) is located within the first 50 nucleotides, preferably within the first 40 nucleotides, more preferably within the first 30 nucleotides, most preferably within the first 20 nucleotides of the 5′-end.

In yet another embodiment, the single stranded oligonucleotide (1) further comprises a primer-annealing element.

In a further embodiment, the primer-annealing element of the single stranded oligonucleotide (1) is complementary to a DNA oligonucleotide (4) attached to a solid support (5), such as a glass slide.

In an embodiment, the oligonucleotide (4) attached to a solid support (5) is modified with an amine, preferably in the 5′-end.

In an embodiment, the primer annealing element of the single stranded oligonucleotide (1) is at least 25 nucleotides long, preferable at least 20 nucleotides long, more preferably at least 18 nucleotides long.

In an embodiment, the single stranded DNA oligonucleotide (1) has a length in the range 80-120 nucleotides, such as 90-110, preferably in the range 95-105 nucleotides, such as 98-102.

In yet an embodiment, the single stranded oligonucleotide (1) further comprises an identification element.

In an embodiment, the identification element of the single stranded oligonucleotide (1) is identical to the sequence of a fluorescently labelled probe (7), said probe preferably being SEQ ID NO: 12.

In a preferred embodiment, the single stranded oligonucleotide (1) is selected from the group consisting of SEQ ID NO: 6-9.

In another preferred embodiment, the single stranded oligonucleotide (1) is free from a sequence element, after position 25 when counted from the 5′-end, selected from the group consisting of:

CTCTT;
GCTTG;
GCTTC;
TCTTG
TCTTC;

and
combinations thereof.

In an embodiment, the subject is a human subject suspected of being infected with Mycobacterium tuberculosis.

### Kit of Parts

The invention also relates to a kit of parts, such as a diagnostic kit. Thus, an aspect of the invention relates to a kit of parts comprising:
a first container comprising a single stranded oligonucleotide (1) according to the invention or a composition according to the invention; and
optionally, instructions for performing a method according to the invention.

In an embodiment, the kit of parts further comprises:
a container comprising a primer sequence (4), such as selected from SEQ ID NO: 10-11, optionally coupled to a solid support (5); and/or
a container comprising at least one detection probe (7), such as SEQ ID NO: 12; and/or
dNTPs, such as comprising labelled dUTPs; and/or
buffers, preferably comprising Mg²⁺ and Mn²⁺ and/or
a polymerase capable of performing rolling circle amplification, preferably being phi29.

In an embodiment, the primer (4) is coupled to a solid surface such as a glass slide.

In an embodiment, the primer (4) is coupled to a solid surface, such as a glass slide and the single stranded DNA oligonucleotide (1) of the composition is hybridized to the primer (4).

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1—Materials and Methods

### Brief Description of Method

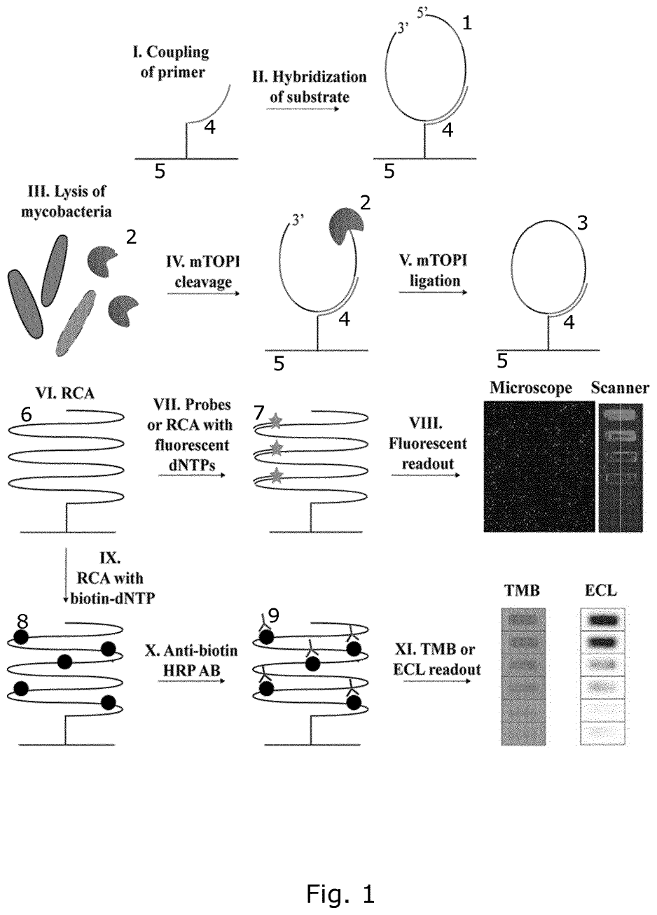

In the section below, the numbering in brackets refers to the numbering in FIG. 1 for overview purposes. The method, named TB-EAD, (FIG. 1) uses a functionalized glass slide (5) coupled to an amine-modified RCA primer (4) (step I). The mTOPI substrate (1) is hybridized to this substrate (step II). The mycobacteria are lysed (step III) and added to the substrate. mTOPI (2) from the extract will react on the STS in the substrate and convert it to a circular product (3) (step IV and V). This product can be amplified by RCA (6) from the RCA primer (4) (step VI). The resulting product can be visualized in a fluorescence microscope or with a fluorescent scanner by using fluorescently labelled probes (7) or incorporating fluorescent nucleotides (step VII and VIII), or with a colorimetric readout by incorporating biotin labelled nucleotides (8) (step IX) that can be coupled to an anti-biotin HRP conjugated antibody (9) (step X) which then can convert the substrate TMB to a visual blue/purple color or ECL into a chemiluminescent reaction that can be visualized with a CCD camera (step XI).

Coupling of RCA primer to Codelink slides:

Codelink slides modified with NHS esters (Surmodics) are divided into squares of 5×5 mm with a mini Pap pen (Thermo Fischer) or with a silicone grid (Grace Biolab). 4 μL containing 50 mM sodium-phosphate buffer, pH 8.5 and 5 UM of the RCA primer (sequence ID 10 or 11, depending on the substrate) is added to each square and coupled to the slide in a humidity chamber containing saturated NaCl for 16-48 hours at room temperature. The slides are then blocked 30 minutes in 50 mM Tris, 50 mM Tris-HCl and 32 mM Ethanolamine pH 9 and washed 2×1 minute in ddH₂O. The slide is finally washed 30 minutes in 4×SSC and 0.1% SDS, 2×1 minute in ddH₂O and left to air-dry before use.

Extraction of the Mycobacteria complex in saliva

Extraction of the Mycobacteria complex in saliva was performed by two times repeated freezing and thawing followed by three times vortex with glass beads for two minutes. The freezing and thawing step was important to obtain sufficient signals to allow for detection of mycobacteria in infected individuals. This is not obvious since vortex with glass beads is a harsh mechanical lysis method that perform well for various prokaryotic and eukaryotic microbes with hard-to-break cell walls (such as gram positive bacteria and budding yeast) and is used as a standard lysis protocol for such organisms in cases where it is important to preserve e.g. enzyme activities.

### TB-EAD:

4 μL containing 10 μmol of the mTOPI substrate in total (SEQ ID NO: 1-9), 10 mM Tris 7.5, 1 mM EDTA and 200 mM NaCl is added to the squares on the slide for 1 hour in a humidity chamber with ddH₂O at 37° C. The slides are washed 1 minute in wash buffer 2 (100 mM Tris-HCl pH 7.5, 150 mM NaCl, 0.3% SDS), 1 minute in wash buffer 3 (100 mM Tris-HCl pH 7.5, 150 mM NaCl, 0.05% Tween20) and 1 minute in 70% ethanol.

200 ng purified mTOPI or 33% v/v mycobacterial extract is added in 10 mM Tris-HCl PH 7.5, 10 mM MgCl2, 10 mM MnCl2, 200 mM NaCl, 1 mM DTT, 0.1% Tween20, 100 μg/mL BSA for 1.5-3 hours in a humidity chamber with ddH2O at 37° C. The slides are washed in wash buffer 2, 3 and 70% ethanol.

RCA is performed for 1-3 hours in 50 mM Tris-HCl PH 7.5, 10 mM MgCl2, 10 mM (NH₄)₂SO₄, 4 mM DTT, 0.2 μg/μL BSA, 250 UM dNTP, 1 Unit/μL Phi29 DNA polymerase (VPCIR Bioscience ApS) in a humidity chamber with ddH2O at 37° C. followed by wash buffer 2, 3 and 70% ethanol.

### Readout Methods:

Detection of Signals with Fluorescent Probes:

2 μmol ID33 detection probe (SEQ ID NO: 12) is added in 2×SSC, 20% formamide and 5% glycerol 30 minutes in a humidity chamber with ddH₂O at 37° C. The slides are washed 10 minutes in wash buffer 2, 5 minutes in wash buffer 3 and 1 minute in 70% ethanol. The slides are mounted with Vectashield (Vector laboratories) and a cover glass and analyzed with a fluorescence microscope at 60× magnification. 10-15 images are acquired for each sample and signals are quantified using the free software ImageJ Fiji.

Detection of signals with fluorescent dUTPs: RCA is performed for 1-3 hours in 50 mM Tris-HCl PH 7.5, 10 mM MgCl2, 10 mM (NH₄)₂S0₄, 4 mM DTT, 0.2 μg/μL BSA, 250 UM dNTP, 125 UM ATTO-488-dUTP (Jena Biosciences), 1 Unit/μL Phi29 DNA polymerase in a humidity chamber with ddH2O at 37° C. followed by wash buffer 2, 3 and 70% ethanol. The slides are washed 10 minutes in wash buffer 2, 5 minutes in wash buffer 3 and 1 minute in 70% ethanol. The slides are mounted with Vectashield (Vector laboratories, USA) and a cover glass and analyzed with a fluorescence microscope at 60× magnification. 10-15 images are acquired for each sample and signals are quantified using the free software ImageJ Fiji. Alternatively, the fluorescent signals can be scanned with a fluorescent scanner when using fluorescent dUTPs and analyzed in ImageJ Fiji.

### Colorimetric Detection of Signals:

RCA is performed for 2 hours with Phi29 DNA polymerase in 50 mM Tris-HCl pH 7.5, 10 mM MgCl2, 10 mM (NH4)2S04, 4 mM DTT, 0.2 μg/μL BSA, 100 μM dATP, 100 μM dGTP, 100 μM TTP, 90 UM dCTP, 10 UM biotin dCTP (Jena Bioscience), and 1 Unit/μL phi29 DNA in a humidity chamber with ddH2O at 37° C. The reaction is stopped by washing for 10 minutes in wash buffer 2, 5 minutes in wash buffer 3 and 1 minute in 70% ethanol. The slide is then blocked in 1×TBST (20 mM Tris-HCl pH 9, 150 mM NaCl, 0.05% Tween20) with 5% BSA and 5% nonfat dry milk. The slide is washed twice in TBST and then subjected to anti-biotin HRP antibody in a 1:50-1:300 dilution with TBST, 5% BSA and 5% nonfat dry milk. The slide is finally washed 3×3 minutes in TBST. 2 μL TMB (Surmodics) is added to each sample and the colorimetric reaction is documented by eye or by taking a picture with a smartphone which then can be used for quantification using ImageJ Fiji.

### Example 2—Detection of TB in Saliva Samples

### Aim of Study

The aim of this study was to evaluate the performance of TB-EAD using saliva samples collected in Guinea-Bissau, West Africa.

Other methods in use uses sputum as test material as it contains a higher number of mycobacteria compared to saliva. The presented invented method is anticipated to have a high sensitivity with the use of mTOPI as marker combined with RCA, which makes the method promising for use in saliva, as tested in this study.

### Materials and Methods

### Sample Collection:

Healthy saliva samples were collected in Denmark and suspected TB patient samples were collected at Hospital Follerau in Guinea Bissau when patients came for diagnosis with the GeneXpert MTB/RIF PCR test (Cepheid). NB: GeneXpert test was performed on sputum. Saliva samples were used directly or frozen at −20° C. until analysis.

### Lysis of Mycobacteria in Saliva Samples:

Samples were lysed by 2× freeze/thawing at −80° C. combined with 3×2 minutes vortexing with 2 minutes break with glass beads. The lysate was used directly for the TB-EAD protocols as described in example 1 using the TP RCA primer (SEQ ID NO 10), the mTOPI TP ID33 STS1 substrate (SEQ ID NO: 1) and fluorescent ID33 probes (SEQ ID NO: 12) for visualization.

### Results

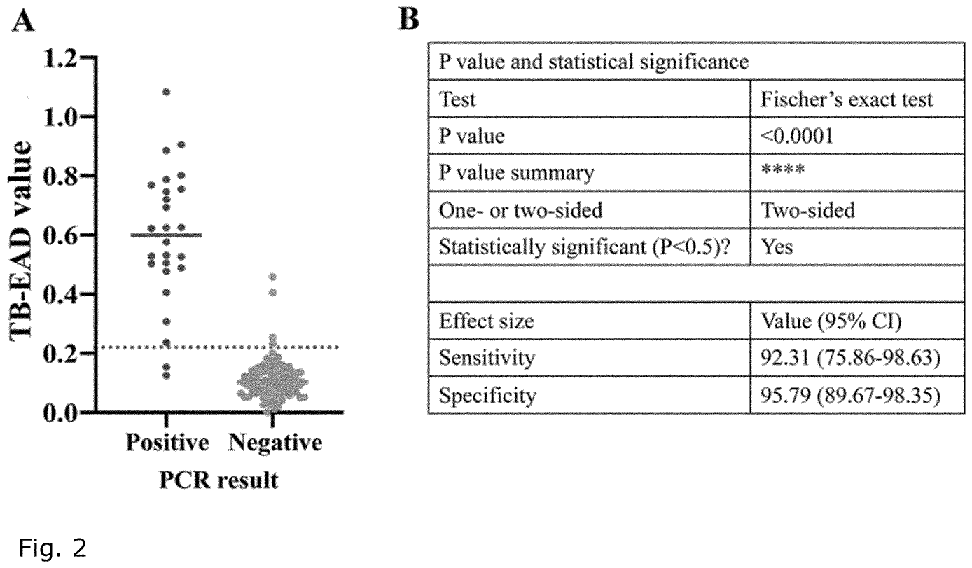

109 saliva samples tested either positive (TB pos) or negative (TB neg) for TB using the GeneXpert MTB/RIF test as standard were analyzed using TB-EAD (FIG. 2A). TB-EAD resulted in a sensitivity and specificity of 92.31% and 95.79%, respectively, when setting threshold for a positive TB infection to 0.22 for relative mTOPI activity. Analyzing the data with a Fischer's exact test results in a significant correlation between the outcomes obtained with TB-EAD compared to GeneXpert (FIG. 2B).

### Conclusion

TB-EAD is able to detect tuberculosis in saliva samples with a sensitivity and specificity of 92.31% and 95.79% when compared to the GeneXpert MTB/RIF PCR test.

### Example 3—Test of Optimized Sequences

### Aim of Study:

The aim of this study was to test whether:

1) increasing the length of the DNA substrate in front of the STS (the 5′ end), 2) including sequences with different STS sequences, and 3) changing the primer and primer binding sequence can increase the number of signals and hence the sensitivity of the method.

### Materials and Methods

Sample preparation: M. smegmatis grown to OD600=0.6 was lysed using a bead-beater with glass beads at 6500 rpm for 3×45 seconds. The extract was used directly for TB-EAD as described in example 1. For aim 1) using the TP RCA primer (SEQ ID NO: 10) and fluorescent ID33 probes (SEQ ID NO: 12) for visualization. The original mTOPI TP ID33 STS1 (SEQ ID NO: 1) was compared to the Long mTOPI TP ID33 STS1 (SEQ ID NO: 2).

For aim 2) using the TP RCA primer (SEQ ID NO: 10) and fluorescent ID33 probes (SEQ ID NO: 12) for visualization. The original mTOPI TP ID33 STS1 (SEQ ID NO: 1) was compared to a mix of substrates with different STS sequences: mTOPI TP ID33 STS1 (SEQ ID NO: 1), mTOPI TP ID33 STS2 (SEQ ID NO: 3), mTOPI TP ID33 STS3 (SEQ ID NO: 4) and mTOPI TP ID33 STS4 (SEQ ID NO: 5).

For aim 3) the mTOPI Mus ID33 STS1 (SEQ ID NO: 6) was tested using the Mus RCA primer (SEQ ID NO: 11) and the fluorescent ID33 probe (SEQ ID NO: 12).

### Oligonucleotide Overview

The differences between SEQ ID Nos: 3-5 and the previously used SEQ ID NO: 1 is the sequences of the STS. The four substrates contain different variations of the STS consensus. The SEQ ID Nos: 6, 7, 8, and 9 are different from SEQ ID NO: 1, 3, 4, and 5 in the primer annealing sequences. This excludes a pseudo STS sequence that may potentially be cleaved by mTOP1. SEQ ID NO: 2 is longer that the original SEQ ID NO: 1 and contains an extended DNA region 5′ to the STS.

### Results

As shown in FIG. 3, increasing the length of the 5′ end of the DNA oligonucleotide does not lead to a much higher number of signals (SEQ ID NO: 2). Including sequences with different STS increases the number of signals around 5-fold (SEQ ID Nos: 1+3+4+5). Testing of the four substrates individually (data not shown) did not result in reproducible results indicating that one or more of them worked better than the others, but the addition of them in combination gave consistent better results than adding them one by one. Using another RCA primer and primer binding sequence in the substrate increases the number around 4-fold (SEQ ID NO: 6).

### Conclusion

A longer DNA substrate, i.e. an extended 5′-end upstream of the STS sequence, does not increase the number of signals. However, including different STS sites and using another primer binding sequence does increase the number of signals. Using the different substrates listed can improve the method by increasing the sensitivity.

The substrates were modified to include all variations of the consensus sequence of the STS. This resulted in four different substrates (SEQ ID Nos: 1, 3, 4, and 5) with each their STS sequence, that each represent one variant of the consensus. Adding a mixture of the four substrates to the assay resulted in increased sensitivity, since it presented the mTOP1 for all variations of the STS consensus.

Also, changing the primer annealing sequence to avoid the inclusion of a pseudo STS (SEQ ID NO: 6, 7, 8, and 9) increased sensitivity, since unspecific cleavage of the primer annealing sequence by mTOP1 could be prevented.

### Example 4—Optimization to One-Step Protocol with Magnesium

### Aim of Study:

Unlike other type IA topoisomerases, mycobacterial TOP1A distinguishes itself from other known enzymes by cleaving single-stranded DNA in an Mg²⁺-independent and sequence specific manner at the strong topoisomerase site (STS). Thus, in a Mg²⁺ depleted buffer, the only enzyme known to be able to cleave and become covalently attached to a DNA substrate is mycobacterial TOPIA. Hence, potential contaminants in a sample that may compromise the specificity of the assay such as other DNA cleaving (e.g. endonucleases, exonucleases) or ligating enzymes can be removed by consecutive wash steps before addition of Mg²⁺, which induces the DNA ligation activity of mycobacterial TOPIA. This two-step approach was utilized by Franch et. Al [1]. The aim of this study was to test whether the TB-EAD method could be improved with a one-step protocol for cleavage and ligation-instead of the two steps as in Franch et al [1].

### Materials and Methods

### Primer Coupling to Codelink Slides:

Primer coupling (slide preparation) and substrate hybridization (using mTOPI TP ID33 (Sequence ID 1)) was performed as described in example 1.

### Lysis:

M. smegmatis, B. megaterium, and L. seeligeri (all gram-positive, tough cell wall) were lysed as described in example 1 with bead-beating. E. coli, E. aerogenes, P. fluorescens (gram negative) were dissolved in a hypotonic buffer and lysed by 60 seconds of vortexing.

mTOPI Reaction—Two-Step Protocol:

For the cleavage reaction 200 ng purified mTOPI or 33% v/v bacterial extract was added to 10 mM Tris-HCl PH 7.5, 200 mM NaCl and 1 mM EDTA for 45 minutes in a humidity chamber with ddH2O at 37° C. The slide was then washed 5 minutes in 10 mM Tris-HCl PH 7.5, 500 mM NaCl and 1 mM EDTA before ligation was performed in 10 mM Tris-HCl PH 7.5, 10 mM MgCl₂, 10 mM MnCl₂, 200 mM NaCl, 1 mM DTT, 0.1% Tween20, 100 μg/mL BSA for 45 minutes in a humidity chamber with ddH2O at 37° C.

mTOPI Reaction—One-Step Protocol:

200 ng purified mTOPI or 33% v/v bacterial extract was added to 10 mM Tris-HCl pH 7.5, 10 mM MgCl₂, 10 mM MnCl₂, 200 mM NaCl, 1 mM DTT, 0.1% Tween20, 100 μg/mL BSA for 1.5 hours in a humidity chamber with ddH2O at 37° C.

### Results

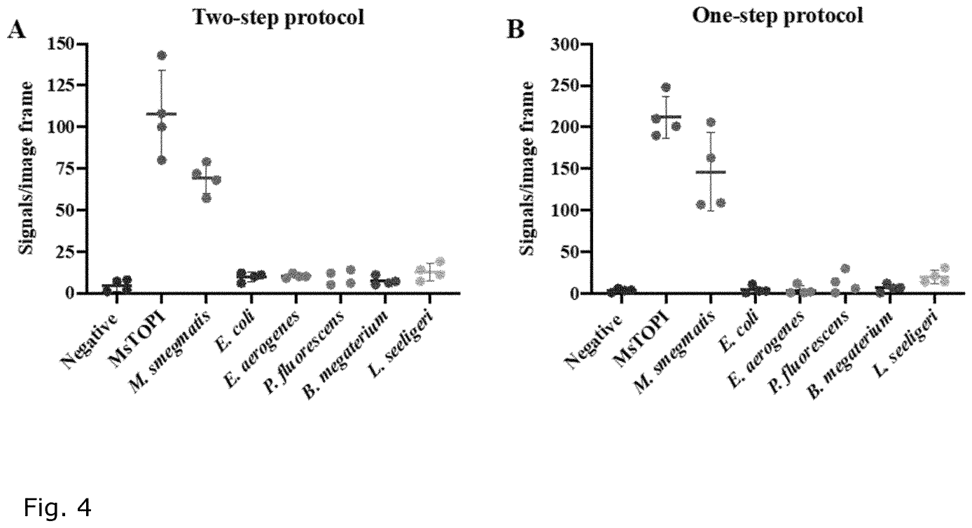

The signals/image frame for the buffer alone (negative), purified M. smegmatis Topoisomerase I (mTOPI), M. smegmatis, E. coli, E. aerogenes, P. fluorescens, B. megaterium and L. seeligeri is comparable between the two-step (FIG. 4A) and one-step protocol (FIG. 4B). Surprisingly, the one-step protocol even increased the sensitivity of the method without compromising the specificity. In addition, the one-step protocol was simpler and faster than the two-step protocol and was therefore applied in the other examples.

### Conclusion

The one-step protocol was simpler, faster, and increased the sensitivity of the method without compromising the specificity.

### Example 5—Optimization of the Lysis Protocols

### Aim of Study:

The aim of this study was to optimize the lysis protocol using M. smegmatis and M. tuberculosis and patient samples.

### Materials and Methods

Coupling of primer (TP RCA primer, sequence ID 10), substrate hybridization (mTOPI TP ID33, sequence ID 1), mTOPI reaction, RCA and coupling of fluorescent detection probe (ID33 probe, sequence ID 12) was performed as described in example 1.

### Lysis:

In all cases, the M. smegmatis were dissolved to 66,000 mycobacteria/μL and M. tuberculosis were dissolved to 266,000 mycobacteria/μL, and glass beads were added in a 1:1 volume ratio.

Bead-beating: 3×45 seconds at 6500 rpm using a tissue homogenizer. The bead-beeting lysis protocol was not tested for M. tuberculosis as machined were not accessible in the biosafety level laboratory.

Vortexing: 3×2 minutes of vortexing or 8×2 minutes of vortexing as inducated in FIG. 5 or 6.

Vortexing combined with freezing and thawing (F/T): Samples were frozen and thawed twice at −60° C. to −80° C. or using liquid nitrogen for 1-10 minutes (the sample needs to be frozen) and then vortexed for 3×2 minutes.

Mycobacteriophages: 1×106 PFU/μL Mycobacteriophages D29 were added for 2 hours at 37° C.

### Results

Lysis of M. smegmatis can be performed with all methods tested in FIG. 5A, however, bead-beating appears to be more effective than 3×2 minutes vortexing or incubating the samples with D29 mycobacteriophages. Using the bacteriophage D29 alone to lyse the M. tuberculosis samples appears to be rather ineffective whereas using vortexing alone or in combination with the bacteriophage D29 is more effective (FIG. 5B). However, the most effective protocol for lysis of M. tuberculosis is freeze/thawing combined with vortexing, preferably for 8×2 minutes (FIG. 5B).

The relative mTOPI activity in the samples without lysis (FIG. 6C) represent the limit for no mTOPI activity. A higher relative mTOPI activity was observed for the Guinea Bissau (GB) patient samples lysed with freeze-thawing and 2×3 minutes vortexing as compared to only 2×3 minutes vortexing. It is therefore clear that the lysis performed using freeze-thawing and 3×2 minutes vortexing (FIG. 6A) is more effective than lysis performed with only 2×3 minutes vortexing (FIG. 6B) i.e. freeze-thawing is an essential step in the effective lysis of mycobacteria in patient samples.

### Conclusion

Freeze/thawing combined with vortexing appears to be the most effective protocol for lysis of M. tuberculosis samples whereas bead-beating was rather effective for lysis of M. smegmatis.

### REFERENCES

[1] Franch O, Han X, Marcussen B, et al. A new DNA sensor system for specific and quantitative detection of mycobacteria. Nanoscale 2019; 2:587-597.

### SEQUENCE LISTING

| SEQ | | |
|---|---|---|
| ID | | |
| NO: | Name* | Sequence** |
| 1 | mTOPI TP ID33 STS 1 | 5′-CAGTGAGCGAGCTTCCGCTTGACATCCCAATATCTCTACT 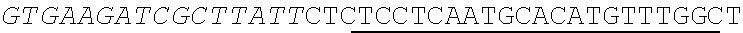 CCTCTCTGAGCTTCCGCT-3′ |
| 2 | Long mTOPI TP ID33 STS 1 | 5′-TGCTGTAACAGTGAGCGAGCTTCCGCTTGACATCCCAATA 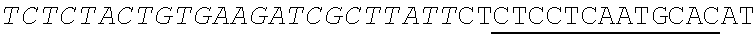 GTTTGGCTCCTCTGAGCTTCCGCT-3′ |
| 3 | mTOPI TP ID33 STS 2 | 5′-CAGTGAGCGAGCTTCCGCTTCACATCCCAATATCTCTACT 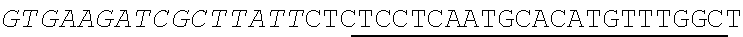 CCTCTCTGAGCTTCCGCT-3′ |
| 4 | mTOPI TP ID33 STS 3 | 5′-CAGTGAGCGAGCTTCCTCTTGACATCCCAATATCTCTACT 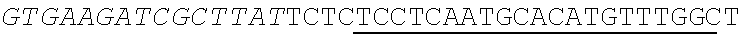 CCTCTCTGAGCTTCCGCT-3 |
| 5 | mTOPI TP ID33 STS 4 | 5′-CAGTGAGCGAGCTTCCTCTTCACATCCCAATATCTCTACT 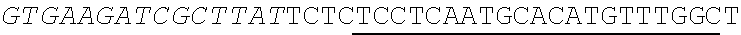 CCTCTCTGAGCTTCCGCT-3′ |
| 6 | mTOPI mus ID33 STS 1 | 5′-CAGTGAGCGAGCTTCCGCTTGACATCCCAATATCTCTACT 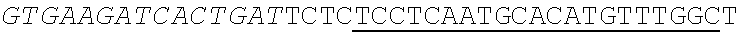 CCTCTCTGAGCTTCCGCT-3′ |
| 7 | mTOPI mus ID33 STS 2 | 5′-CAGTGAGCGAGCTTCCGCTTCACATCCCAATATCTCTACT 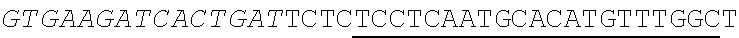 CCTCTCTGAGCTTCCGCT-3′ |
| 8 | mTOPI mus ID33 STS 3 | 5′-CAGTGAGCGAGCTTCCTCTTGACATCCCAATATCTCTACT 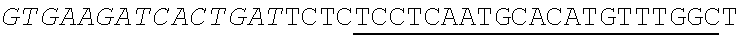 CCTCTCTGAGCTTCCGCT-3 |
| 9 | mTOPI mus ID33 STS 4 | 5′-CAGTGAGCGAGCTTCCTCTTCACATCCCAATATCTCTACT 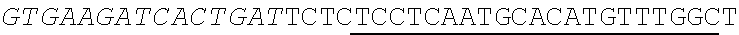 CCTCTCTGAGCTTCCGCT-3′ |
| 10 | TP RCA | 5′-Amine-CCAACCAACCAACCAA- |
| | primer | ATAAGCGATCTTCACAGT-3′ |
| 11 | Mus RCA | 5′-Amine-CCAACCAACCAACCAA- |
| | primer | ATCAGTGATCTTCACAG-3′ |
| 12 | ID33 probe | 5′-FAM-CCTCAATGCACATGTTTGGCTCC-3′ |

*Note that mTOPI TP substrates should be used with the TP RCA primer, while the mTOPI mus substrates should be used with the mus RCA primer,
**Bold marks STS;
Underling indicates ID33 probe binding site after rolling circle amplification;
Italic indicates primer-binding site for either the TP RCA primer or the MUS RCA primer;
Gray boxes indicate the site of an identified pseudo STS site in SEQ ID NO's: 1-5, said site has been removed in SEQ ID NO′s: 6-9.

### Items

1. A method for determining whether a subject is likely to be infected with Mycobacterium tuberculosis, the method comprising
a) having a (previously obtained) saliva sample from a subject provided;
b) providing one or more types of single stranded DNA oligonucleotides (1) comprising:
   a binding element (Strong Topoisomerase Site; STS) for Mycobacterium tuberculosis DNA Topoisomerase I enzyme (mTOPI) (2);
c) incubating the saliva sample of step a) with the one or more types of oligonucleotides (1) from step b), wherein if mTOPI (2) is present in the sample of step a), the one or more types of single stranded DNA oligonucleotides of step b) are circularized (3);
d) determining the level of circularized oligonucleotides in the sample from step c);
e) comparing said determined level to a reference level; and
f) determining that
   if said level is above said reference level it is indicative of said subject being infected with Mycobacterium tuberculosis; or
   if said reference level is equal to or below said reference, it is indicative of said subject not being infected with Mycobacterium tuberculosis.

2. The method according to item 1, wherein the subject is a mammal preferably a human.

3. The method according to any one of items 1 or 2, wherein the saliva sample of step a) is a lysed saliva sample.

4. The method according to item 3, wherein the lysis is performed by i) freeze and thawing the saliva sample, preferably followed by vortexing with a solid material, such as glass beads; ii) bead beating; and/or iii) using lytic mycobacteriophages.

5. The method according to any preceding item, wherein the binding element of the one or more types of single stranded oligonucleotides (1) of step b) is selected from the group consisting of 5′-CGCTTG-3′, 5′-CGCTTC-3′, 5′-CTCTTG-3′, and 5′-CTCTTC-3′.

6. The method according to any of the preceding items, wherein in step b) a combinations of types of single stranded oligonucleotides (1) are used, comprising different binding elements selected from 5′-CGCTTG-3′, 5′-CGCTTC-3′, 5′-CTCTTG-3′, and 5′-CTCTTC-3′.

7. The method according to any of the preceding items, wherein two different types of single stranded oligonucleotides (1) are used, preferably three different types of single stranded oligonucleotides (1) and more preferably four different types of single stranded oligonucleotides (1).

8. The method according to any of the preceding items, wherein the binding element of the one or more types of single stranded oligonucleotides (1) of step b) is located within the first 50 nucleotides, preferably within the first 40 nucleotides, more preferably within the first 30 nucleotides, most preferably within the first 20 nucleotides of the 5′-end.

9. The method according to any of the preceding items, wherein the one or more types of single stranded oligonucleotide (1) of step b) further comprises a primer-annealing element.

10. The method according to item 9, wherein the primer-annealing element of the one or more types of single stranded oligonucleotides (1) of step b) is complementary to a DNA oligonucleotide (4) attached to a solid support (5), such as a glass slide.

11. The method according to item 10, wherein the oligonucleotide (4) attached to a solid support (5) is modified with an amine, preferably at the 5′-end.

12. The method according to any one of items 9-11 wherein the primer annealing element of the one or more single stranded oligonucleotides (1) of step b) is at least 25 nucleotides long, preferable at least 20 nucleotides long, more preferably at least 18 nucleotides long.

13. The method according to any of the preceding items, wherein the one or more types of single stranded DNA oligonucleotides (1) of step b) has a length in the range 80-120 nucleotides, such as 90-110, preferably in the range 95-105 nucleotides, such as 98-102.

14. The method according to any of the preceding items, wherein the one or more types of single stranded oligonucleotides (1) of step b) further comprises an identification element.

15. The method according to item 14, wherein the identification element of the one or more types of single stranded oligonucleotides (1) of step b) is identical to the sequence of a fluorescently labelled probe (7), said probe preferably being SEQ ID NO: 12.

16. The method according to any of the preceding items, wherein the one or more types of single stranded oligonucleotides (1) of step b) is selected from the group consisting of SEQ ID NO: 1-9 or combinations thereof, such as selected from SEQ ID NO: 2-6 or combinations thereof, or selected from SEQ ID NO: 3-9 or combinations thereof, preferably selected from SEQ ID NO: 3-6 or combinations thereof.

17. The method according to any of the preceding items, wherein the one or more types of single stranded oligonucleotides (1) of step b) is selected from the group consisting of SEQ ID NO: 3-9 or combinations thereof.

18. The method according to any of the preceding items, wherein the one or more types of single stranded oligonucleotides (1) is free of a 5′-CGCTT-3′ sequence element after position 25, when counted from the 5′-end, such as after position 30 or after position 35.

19. The method according to any of the preceding items, wherein the one or more types of single stranded oligonucleotides (1) is free from one or more sequence elements, after position 25 when counted from the 5′-end, selected from the group consisting of:

CGCTT;
CTCTT;
GCTTG;
GCTTC;
TCTTG; and
TCTTC.

20. The method according to any of the preceding items, wherein in step c) Mg²⁺ ions are present, such as at a concentration in the range 2-20 mM, preferably 5-15 mM, more preferably 8-12 mM.

21. The method according to any of the preceding items, wherein in step c) Mn²⁺ ions are present, such as at a concentration in the range 2-20 mM, preferably 5-mM, more preferably 8-12 mM.

22. The method according to any of the preceding items, wherein the level of circularized DNA (3) of step d) is determined using a method selected from the group consisting of Rolling Circle Amplification (RCA), PCR, real-time-PCR, Southern blotting, quantitative PCR (qPCR), restriction fragment length dimorphism-PCR (RFLD-PCR), primer extension, DNA array technology, LAMP and isothermal amplification, preferably using RCA (6).

23. The method according to item 22, wherein a portion of the dNTPs, such as all dUTPs, are modified.

24. The method according to item 23, wherein the portion of dNTPs being modified, are modified by the addition of a fluorescent label or a biotin group (8).

25. The method according to any of the preceding items, wherein the reference level of step e) is determined in a subject or a group of subjects without a Mycobacterium tuberculosis infection.

26. The method according to any of the preceding items, wherein the reference level of step e) is determined using the same method as used to determine the level of circularized DNA in step d).

27. An isolated single stranded DNA oligonucleotide (1) comprising:
a binding element (Strong Topoisomerase Site; STS) for Mycobacterium tuberculosis DNA Topoisomerase I enzyme (mTOPI) (2) selected from the group consisting of 5′-CGCTTG-3′, 5′-CGCTTC-3′, 5′-CTCTTG-3′, and 5′-CTCTTC-3′, said binding element being located within the first 30 nucleotides, preferably within the first 20 nucleotides of the 5′-end;
optionally, a primer-annealing element located after the binding element, when counted from the 5′-end, said primer-annealing element being complementary to a DNA oligonucleotide (4) attached to a solid support (5), such as a glass slide;
optionally, an identification element identical to the sequence of a fluorescently labelled probe (7), said probe preferably being SEQ ID NO: 12;
   wherein the single stranded oligonucleotide (1) is free of a 5′-CGCTT-3′ sequence element after position 25, when counted from the 5′-end, such as after position 30 or after position 35.

28. The isolated single stranded DNA oligonucleotide (1) according to item 24, wherein said single stranded DNA oligonucleotide (1) has a length in the range of 80-120 nucleotides, such as 90-110, preferably in the range of 95-105 nucleotides, such as 98-102.

29. The isolated single stranded DNA oligonucleotide according to any of items 27-28, wherein said oligonucleotide is selected from the group consisting of SEQ ID NO. 6-9.

30. A composition comprising two or more types of single stranded oligonucleotides (1) according to any of items 27-29; wherein the two or more types have different binding elements (Strong Topoisomerase Site; STS) for Mycobacterium tuberculosis DNA Topoisomerase I enzyme (mTOPI) (2) selected from the group consisting of 5′-CGCTTG-3′, 5′-CGCTTC-3′, 5′-CTCTTG-3′, and 5′-CTCTTC-3′.

31. The composition according to item 30, comprising three or more types of single stranded oligonucleotides (1) according to any of items 27-29; wherein the three or more types have different binding elements (Strong Topoisomerase Site; STS) for Mycobacterium tuberculosis DNA Topoisomerase I enzyme (mTOPI) (2) selected from the group consisting of 5′-CGCTTG-3′, 5′-CGCTTC-3′, 5′-CTCTTG-3′, and 5′-CTCTTC-3′.

32. The composition according to any of items 30-31, comprising four types of single stranded oligonucleotides (1) according to any of items 27-29; wherein the four types have different binding elements (Strong Topoisomerase Site; STS) for Mycobacterium tuberculosis DNA Topoisomerase I enzyme (mTOPI) (2) selected from the group consisting of 5′-CGCTTG-3′, 5′-CGCTTC-3′, 5′-CTCTTG-3′, and 5′-CTCTTC-3′.

33. The composition according to any of items 30-32, wherein the ratio (by concentration) between the two or more single stranded oligonucleotides (1) deviates at the most by 20% from the one with the highest concentration to the one with the lowest concentration, such as deviating at the most by 10%, such as by the most 5%, preferably the concentration for each oligonucleotide is the same.

34. Use of one or more single stranded oligonucleotides (1) according to any one of items 27-29 or a composition according to any of items 30-33 for determining, in a saliva sample, whether a subject is likely to be infected with Mycobacterium tuberculosis (mt).

35. Use of a single stranded DNA oligonucleotide (1) comprising a binding element (Strong Topoisomerase Site; STS) for Mycobacterium tuberculosis DNA Topoisomerase I enzyme (mTOPI) (2) for determining, in a saliva sample, whether a subject is likely to be infected with Mycobacterium tuberculosis (mt).

36. The use according to item 35, wherein the STS is selected from the group consisting of 5′-CGCTTG-3′, 5′-CGCTTC-3′, 5′-CTCTTG-3′, and 5′-CTCTTC-3′.

37. The use according to any of items 34-36, wherein the STS of the single stranded oligonucleotide (1) is located within the first 50 nucleotides, preferably within the first 40 nucleotides, more preferably within the first 30 nucleotides, most preferably within the first 20 nucleotides of the 5′-end.

38. The use according to any of items 34-37, wherein the single stranded oligonucleotide (1) further comprises a primer-annealing element.

39. The use according to item 38, wherein the primer-annealing element of the single stranded oligonucleotide (1) is complementary to a DNA oligonucleotide (4) attached to a solid support (5), such as a glass slide.

40. The use according to item 39, wherein the oligonucleotide (4) attached to a solid support (5) is modified with an amine, preferably in the 5′-end.

41. The use according to any of items 34-40, wherein the primer annealing element of the single stranded oligonucleotide (1) is at least 25 nucleotides long, preferable at least 20 nucleotides long, more preferably at least 18 nucleotides long.

42. The use according to any of items 34-41, wherein the single stranded DNA oligonucleotide (1) has a length in the range 80-120 nucleotides, such as 90-110, preferably in the range 95-105 nucleotides, such as 98-102.

43. The use according to any of items 34-42, wherein the single stranded oligonucleotide (1) further comprises an identification element.

44. The use according to item 43, wherein the identification element of the single stranded oligonucleotide (1) is identical to the sequence of a fluorescently labelled probe (7), said probe preferably being SEQ ID NO: 12.

45. The use according to any of items 34-44, wherein the single stranded oligonucleotide (1) is selected from the group consisting of SEQ ID NO: 6-9.

46. Use of the single stranded DNA oligonucleotide (1) according to any one of items 34-45, wherein the single stranded oligonucleotide (1) is free from one or more sequence elements, after position 25 when counted from the 5′-end, selected from the group consisting of:

CGCTT;
CTCTT;
GCTTG
GCTTC;
TCTTG; and
TCTTC.

47. Use of an oligonucleotide according to any one of items 34-46, wherein the subject is a human subject suspected of being infected with Mycobacterium tuberculosis.

48. A kit of parts comprising:
a first container comprising a DNA oligonucleotide (1) according to any one of items 27-29 or a composition according to any of items 30-33; and
optionally, instructions for performing a method according to any of items 1-26.

49. The kit of parts according to item 48 further comprising:
a container comprising a primer sequence (4), such as selected from SEQ ID NO: 10-11, optionally coupled to a solid support (5); and/or
a container comprising at least one detection probe (7), such as SEQ ID NO: 12; and/or
dNTPs, such as comprising labelled dUTPs; and/or
buffers; and/or
a polymerase capable of performing rolling circle amplification, preferably being phi29.

50. The kit of parts according to item 49, wherein the primer is coupled to a solid surface, such as a glass slide.

1. A method for determining whether a subject is likely to be infected with Mycobacterium tuberculosis, the method comprising
a) having a saliva sample from a subject provided;
b) providing a combination of types of single stranded DNA oligonucleotides comprising:
a binding element (Strong Topoisomerase Site; STS) for Mycobacterium tuberculosis DNA Topoisomerase I enzyme (mTOPI);
a primer-annealing element, complementary to a DNA oligonucleotide;
wherein the single stranded DNA oligonucleotides are attached to a solid support via the DNA oligonucleotide;
c) incubating the saliva sample of step a) with the oligonucleotides from step b), wherein if mTOPI is present in the sample of step a), the single stranded DNA oligonucleotides of step b) are circularized;
d) determining the level of circularized oligonucleotides in the sample from step c);
e) comparing said determined level to a reference level; and
f) determining that
if said level is above said reference level it is indicative of said subject being infected with Mycobacterium tuberculosis; or
if said reference level is equal to or below said reference, it is indicative of said subject not being infected with Mycobacterium tuberculosis;
wherein in step b) the types of single stranded oligonucleotides (1) comprise different binding elements selected from 5′-CGCTTG-3′, 5′-CGCTTC-3′, 5′-CTCTTG-3′, and 5′-CTCTTC-3′;
wherein in step c) Mg²⁺ ions are present;
wherein in step d) the level of circularized DNA of step d) is determined using a method selected from the group consisting of Rolling Circle Amplification (RCA), PCR, real-time-PCR, Southern blotting, quantitative PCR (qPCR), restriction fragment length dimorphism-PCR (RFLD-PCR), primer extension, DNA array technology, LAMP, and isothermal amplification.

2. The method according to claim 1, wherein the binding element of the types of single stranded oligonucleotides of step b) is selected from the group consisting of 5′-CGCTTG-3′, 5′-CGCTTC-3′, 5′-CTCTTG-3′, and 5′-CTCTTC-3′.

3. The method according to claim 1, wherein the types of single stranded DNA oligonucleotides (1) of step b) has a length in the range 80-120 nucleotides.

4. The method according to claim 1, wherein the types of single stranded oligonucleotides of step b) is selected from the group consisting of SEQ ID NO: 1-9 or combinations thereof.

5. The method according to claim 1, wherein the types of single stranded oligonucleotides (1) of step b) is selected from the group consisting of SEQ ID NO: 3-9 or combinations thereof.

6. The method according to claim 1, wherein the types of single stranded oligonucleotides is free of a 5′-CGCTT-3′ sequence element after position 25, when counted from the 5′-end.

7. The method according to claim 1, wherein the types of single stranded oligonucleotides is free from one or more sequence elements, after position 25 when counted from the 5′-end, selected from the group consisting of:
CGCTT;
CTCTT;
GCTTG;
GCTTC;
TCTTG; and
TCTTC.

8. The method according to claim 1, wherein in step c) Mg²⁺ ions are present at a concentration in the range 2-20 mM.

9. A composition comprising two or more types of single stranded oligonucleotides comprising
a binding element (Strong Topoisomerase Site; STS) for Mycobacterium tuberculosis DNA Topoisomerase I enzyme (mTOPI) (2) selected from the group consisting of 5′-CGCTTG-3′, 5′-CGCTTC-3′, 5′-CTCTTG-3′, and 5′-CTCTTC-3′, said binding element being located within the first 30 nucleotides, preferably within the first 20 nucleotides of the 5′-end;
a primer-annealing element located after the binding element, when counted from the 5′-end, said primer-annealing element being complementary to a DNA oligonucleotide attached to a solid support;
wherein the single stranded oligonucleotides are free of a 5′-CGCTT-3′ sequence element after position 25, when counted from the 5′-end, such as after position 30 or after position 35;
wherein the two or more types have different binding elements (Strong Topoisomerase Site; STS) for Mycobacterium tuberculosis DNA Topoisomerase I enzyme (mTOPI) (2) selected from the group consisting of 5′-CGCTTG-3′, 5′-CGCTTC-3′, 5′-CTCTTG-3′, and 5′-CTCTTC-3′.

10. The composition according to claim 9, comprising an identification element identical to the sequence of a fluorescently labelled probe.

11. The composition according to claim 9, comprising three or more types of single stranded oligonucleotides; wherein the three or more types have different binding elements (Strong Topoisomerase Site; STS) for Mycobacterium tuberculosis DNA Topoisomerase I enzyme (mTOPI) selected from the group consisting of 5′-CGCTTG-3′, 5′-CGCTTC-3′, 5′-CTCTTG-3′, and 5′-CTCTTC-3′.

12. The composition according to claim 9, comprising four types of single stranded oligonucleotides; wherein the four types have different binding elements (Strong Topoisomerase Site; STS) for Mycobacterium tuberculosis DNA Topoisomerase I enzyme (mTOPI) selected from the group consisting of 5′-CGCTTG-3′, 5′-CGCTTC-3′, 5′-CTCTTG-3′, and 5′-CTCTTC-3′.

13. The composition according to claim 9, wherein said single stranded DNA oligonucleotide has a length in the range of 80-120 nucleotides.

14. The composition according to claim 9, wherein said oligonucleotides are selected from the group consisting of SEQ ID NOS: 6-9.

15. A kit of parts comprising:
a first container comprising a composition according to claim 9.

16. The kit of parts according to claim 15, further comprising:
a container comprising a primer sequence; and/or
a container comprising at least one detection probe; and/or
dNTPs; and/or
a polymerase capable of performing rolling circle amplification.

17. The kit of parts according to claim 15, comprising a primer sequence coupled to a solid surface.

18. The kit of parts according to any of claim 15, comprising primer sequence coupled to a solid surface and wherein the single stranded DNA oligonucleotide of the composition is hybridized to the primer.

19. The method according to claim 1, wherein in step d) the level of circularized DNA of step d) is determined using Rolling Circle Amplification (RCA).


SEQUENCE LISTING (12 sequences)

SEQ ID NO: 1 - DNA, 99 bp, synthetic construct
  source 1..99: mol_type = other DNA
cagtgagcgagcttccgcttgacatcccaatatctctactgtgaagatcgcttattctctcctcaatgcacatgtttggctcctctctgagcttccgct

SEQ ID NO: 2 - DNA, 105 bp, synthetic construct
  source 1..105: mol_type = other DNA
tgctgtaacagtgagcgagcttccgcttgacatcccaatatctctactgtgaagatcgcttattctctcctcaatgcacatgtttggctcctctgagcttccgct

SEQ ID NO: 3 - DNA, 99 bp, synthetic construct
  source 1..99: mol_type = other DNA
cagtgagcgagcttccgcttcacatcccaatatctctactgtgaagatcgcttattctctcctcaatgcacatgtttggctcctctctgagcttccgct

SEQ ID NO: 4 - DNA, 99 bp, synthetic construct
  source 1..99: mol_type = other DNA
cagtgagcgagcttcctcttgacatcccaatatctctactgtgaagatcgcttattctctcctcaatgcacatgtttggctcctctctgagcttccgct

SEQ ID NO: 5 - DNA, 99 bp, synthetic construct
  source 1..99: mol_type = other DNA
cagtgagcgagcttcctcttcacatcccaatatctctactgtgaagatcgcttattctctcctcaatgcacatgtttggctcctctctgagcttccgct

SEQ ID NO: 6 - DNA, 99 bp, synthetic construct
  source 1..99: mol_type = other DNA
cagtgagcgagcttccgcttgacatcccaatatctctactgtgaagatcactgattctctcctcaatgcacatgtttggctcctctctgagcttccgct

SEQ ID NO: 7 - DNA, 99 bp, synthetic construct
  source 1..99: mol_type = other DNA
cagtgagcgagcttccgcttcacatcccaatatctctactgtgaagatcactgattctctcctcaatgcacatgtttggctcctctctgagcttccgct

SEQ ID NO: 8 - DNA, 99 bp, synthetic construct
  source 1..99: mol_type = other DNA
cagtgagcgagcttcctcttgacatcccaatatctctactgtgaagatcactgattctctcctcaatgcacatgtttggctcctctctgagcttccgct

SEQ ID NO: 9 - DNA, 99 bp, synthetic construct
  source 1..99: mol_type = other DNA
cagtgagcgagcttcctcttcacatcccaatatctctactgtgaagatcactgattctctcctcaatgcacatgtttggctcctctctgagcttccgct

SEQ ID NO: 10 - DNA, 34 bp, synthetic construct
  source 1..34: mol_type = other DNA
  modified_base <1: mod_base = OTHER; note = Amine
ccaaccaaccaaccaaataagcgatcttcacagt

SEQ ID NO: 11 - DNA, 33 bp, synthetic construct
  source 1..33: mol_type = other DNA
  modified_base <1: mod_base = OTHER; note = Amine
ccaaccaaccaaccaaatcagtgatcttcacag

SEQ ID NO: 12 - DNA, 23 bp, synthetic construct
  source 1..23: mol_type = other DNA
  modified_base <1: mod_base = OTHER; note = Fluorescein amidite (FAM)
cctcaatgcacatgtttggctcc